(19) United States
(12) Patent Application Publication
(10) Pub. No.: US 2010/0069494 A1
(43) Pub. Date: Mar. 18, 2010

(54) LOWERING OF PROTEINS ASSOCIATED WITH ALZHEIMER'S DISEASE BY INTERRUPTING GENE TRANSCRIPTION WITH A SMALL MOLECULE

(75) Inventor: Nasser H. Zawia, Warwick, RI (US)

Correspondence Address: GAUTHIER & CONNORS, LLP, 225 FRANKLIN STREET, SUITE 2300, BOSTON, MA, 02110, US

(73) Assignee: BOARD OF GOVERNORS FOR HIGHER EDUCATION, STATE OF RHODE ISLAND AND PROVIDEN, Providence, RI (US)

(21) Appl. No.: 12/570,429

(22) Filed: Sep. 30, 2009

Related U.S. Application Data
(63) Continuation of application No. PCT/US08/59402, filed on Apr. 4, 2008.
(60) Provisional application No. 60/910,493, filed on Apr. 6, 2007.

(51) Int. Cl. A61K 31/195; A61P 25/28

(52) U.S. Cl. 514/567

(57) ABSTRACT

A method of treating Alzheimer's disease in a patient. The method includes administrating tolfenamic acid to the patient to modulate precursors of pothgene intermediates. The treatment also lowers SP1, APP and Aβ1 levels.

### PRIORITY INFORMATION

This application is a continuation of International Patent Application No. PCT/US08/59402, filed on Apr. 4, 2008 which claims priority to U.S. Provisional Patent Application No. 60/910,493, filed on Apr. 6, 2007, all of which are incorporated herein in their entirety.

### BACKGROUND OF THE INVENTION

An estimated 10% of Americans over the age of 65 and half of those over age 85 have Alzheimer's Disease (“AD”). The number of people suffering from this neurodegenerative disease is expected to increase exponentially in the coming decades due to an increasing lifespan and an aging “baby-boomer” generation. AD is the third most expensive disease in the US estimated to cost the US economy about $100 billion annually. Standard therapeutic approaches have failed and there is a need to develop new drugs in order to avoid an “epidemic crisis” in the future.

AD is the most prevalent form of senile dementia. One hundred years after the discovery of AD, the etiology of this disease still remains unknown and although a few drugs have been approved, their benefits have been extremely modest. Advances in basic research have identified many pathways that lead to the formation of key pathological features of AD, Amyloid plaques (amyloidogenesis), and abnormal twisted forms of the protein tau (tangles). The available limited treatment strategies have mostly focused on targets associated with the production of the amyloid (Aβ) peptides which aggregate into various assemblies and their deposits from the core of the plaque.

AD is characterized by chronic and progressive loss of neurons in discrete areas of the brain, causing debilitating symptoms such as dementia, loss of memory, and eventually, premature death. Currently five drugs are approved for AD in the US. With exception of Memantine, all are cholinesterase inhibitors designed to build up the levels of the neurotransmitter acetylcholine, which are low due to the early loss of neurons that contain this neurotransmitter. On the other hand, Memantine prevents over-stimulation of the NMDA type of glutamate receptors which contribute to the pathogenesis of several neurodegenerative diseases. All of the approved drugs are not disease modifying, but have shown very modest improvements in behavioral and functional measures in a subset of patients.

Several drugs are in the pipeline undergoing phase I and II safety studies and phase III clinical studies. The overall emphasis of these drugs has been to ameliorate the pathogenicity of Aβ peptides by reducing Aβ production through inhibiting the secretases that process Aβ, enhancing Aβ clearance by immune mechanisms, and disrupting Aβ aggregation using small molecules. Other investigative drugs target the overall process of neurodegeneration by combating oxidants or preventing cell loss. Furthermore several “off-the-shelf” drugs such as the anti-inflammatory NSAIDS and cholesterol lowering drugs are being tested for their ability to decrease the risk associated with AD through their anti-inflammatory properties.

### SUMMARY OF THE INVENTION

Given the poor performance of existing therapies and low potential for drugs in development, there is an increasing need to develop alternative drugs that modify the disease process. Drugs that have the ability to impact several intermediates associated with AD with one single hit are most desirable

The approach is based on developing drugs that target transcription factors such as specificity protein 1 (Sp1) and thus provide the ability to influence downstream pathways. Studies using rodent and primate brain slices, showed that Sp1 is differentially expressed and found in neurons which are abundant in the Amyloid Precursor Protein (APP) and its amyloidogenic peptide (Aβ). Further published studies have shown that the expression of APP is dependent on Sp1 and others have showed the importance of Sp1 for BACE1 expression, an enzyme that cleaves APP. Thus targeting Sp1 is likely to affect the expression levels of the APP gene and further production of its products which are associated with the pathogenesis of AD.

Tolfenamic acid a non-steroidal anti inflammatory drug (NSAID) can induce proteasome-dependent degradation of Sp1, Sp3 and Sp4 in pancreatic cancer cells and tumors thereby inhibiting tumor growth/angiogenesis. This property is unique for tolfenamic acid and is not exhibited by standard NSAIDS. Therefore, it was hypothesized that tolfenamic acid will induce degradation of Sp1 in the brain and represent a novel class of mechanism-based drugs for the treatment of Alzheimer's disease.

One advantage of this approach is that it utilizes an existing approved drug for a new application in AD. The safety of this drug has already been shown, and the efficacious doses are lower than has been approved for treatment of other diseases. The projected dose in humans is 1-5 mg/kg. The cost of the treatment is low and patients can receive this care outside of a hospital setting. If administered in the early stages of AD, this drug can potentially lower pathogenic endpoints by 25-50%. This drug could prevent as many as 50% of the patients from developing AD.

There is currently no known AD drug that works through this unique mechanism. There is no approved drug that can modulate the precursors of the pathogenic intermediates. All existing approved drugs are not disease modifying but offer only symptomatic relief.

The efficacy of tolfenamic acid in modifying the levels of proteins associated with AD has been tested. It has been found that in mice it lowered cerebral Sp1 and consequentially both APP and Aβ levels. Since the regulation of the APP gene is highly conserved, this drug will also do the same in humans and constitutes a new mechanism-based disease modifying drug that is useful for the treatment of AD.

These and other features and objectives of the present invention will now be described in greater detail with reference to the accompanying drawings, wherein:

### BRIEF DESCRIPTION OF THE DRAWINGS

FIGS. 1A-F are micrographs of SP1, APP and Aβ Immunoreactivity in the Rat Cortex;

FIGS. 2A-F are micrographs of SP1, APP and Aβ immunoreactivity in hippocampus and corpus callosum of rat;

FIGS. 3A-F are micrographs of SP1, APP and Aβ Immunoreactivity in rat cerebellum;

FIGS. 4A-F are photomicrographs of SP1, APP and Aβ Immunoreactivity in Monkey Cortex;

FIG. 5 is a graph of Human APP promoter activity and Gene Silencing (siRNA) in transfected PC12 Cells;

FIGS. 6A is a western blot and FIGS. 6B and 6 are graphs depicting the Oral Administration of Tolfenamic acid to mice which lowers SP1, APP, and Aβ levels in the brain; and

FIG. 7 is a scheme illustrating the knockdown of APP expression and the consequence on AD pathology.

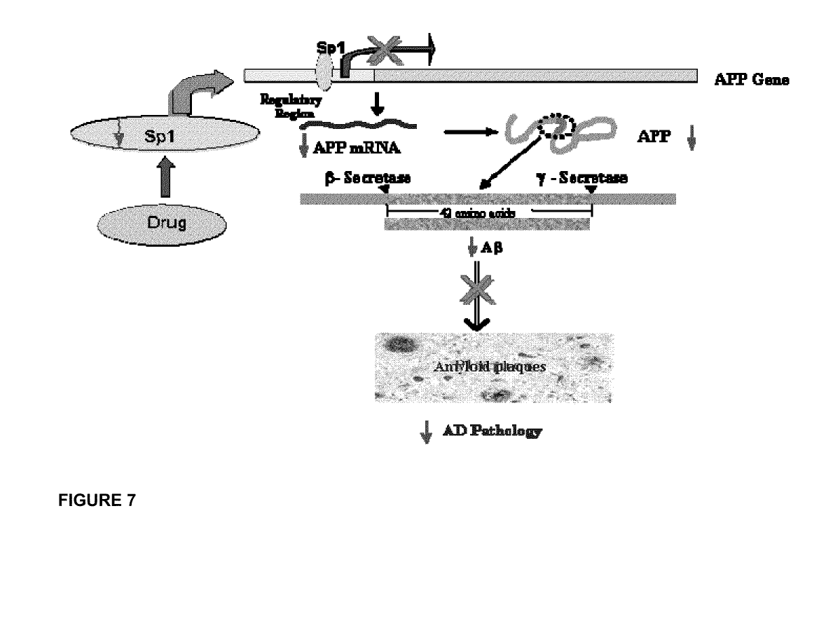

### DESCRIPTION OF THE INVENTION

Tolfenamic acid has efficacy in the treatment of AD by acting through an entirely new mechanism that is independent of its anti-inflammatory properties. This drug which is approved in the United Kingdom for migraine headaches can target the Sp transcription factor family and thus serve as a tool to knock down genes associated with certain diseases. This small molecule accomplishes what cumbersome and non-drugable gene knock-down or knock-out approaches can. This approach of acting upstream at the genome level has the ability to impact several intermediates associated with AD and other neurodegenerative diseases with one single agent.

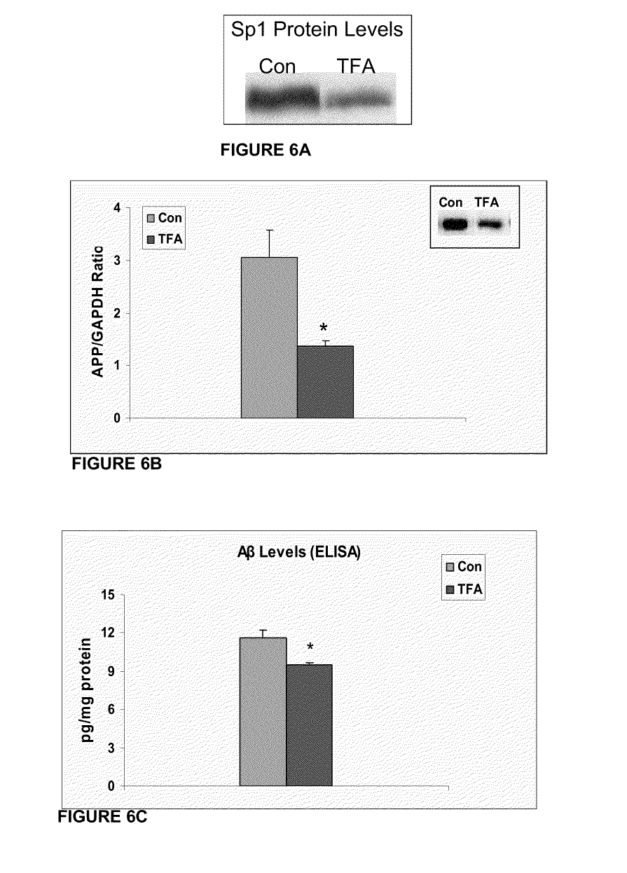

The Oral Administration of Tolfenamic acid to mice lowers SP1, APP, and Aβ levels in the brain (See FIG. 6). Mice were dosed on alternate days by oral gavage with 10 or 50 mg/Kg of tolfenamic acid in corn oil or vehicle alone. Tolfenamic acid (TFA) lowered APP levels by about 50% and Aβ by about 20%. The lower doses were more effective. FIG. 6A is a representative Western blot of SP1. FIG. 6B is a Western blot analysis of APP expressed as a ratio of a housekeeping gene, FIG. 6C is Aβ analysis by ELISA. Each bar represents the mean of data obtained from 6-9 animals. Data from both doses was combined after statistical analysis. Values marked with an ‘*’ are statistically significant, p<0.0.5.

### METHODS

Rats: Timed-pregnant Long-Evans hooded rats were obtained from Charles River Laboratories (Wilmington, Mass.). Day of birth was considered postnatal day 0 (PND0). To randomize prenatal and genetic factors, on PND1, pups from all litters were pooled and new litters were reconstituted by the random selection of 9-10 male pups per litter. Litters were maintained at a constant litter size over the course of lactation with the addition of female filler pups if one of the original male pups died. The animals were housed at an ambient temperature (21±2° C.) and relative humidity (50±10%) with a 12-hour light/dark cycle (0700-1900 hours). Food and water were freely available throughout the study. Pups were weaned on PND21 and placed in group housing (3 pups/cage) for three months and then housed individually. All animal procedures were conducted according to a protocol approved by the Institutional Animal Care and Use

Committee (IACUC) of the University of Rhode Island.

On PND 600 (20 months) 5 animals (one from each litter) were used for the study. These animals were deeply anesthetized with an intraperitoneal injection of sodium pentobarbital (40 mg/kg body weight) and transcardially perfused with 100 cc of perfusion wash (0.8% Sodium Chloride, 0.4% Dextrose, 0.8% Sucrose, 0.023% Calcium chloride and 0.034% anhydrous-Sodium Cacodylate) followed by 100 cc perfusion fix (4% sucrose, 4% paraformaldehyde and 1.07% anhydrous-Sodium Cacodylate, pH 7.2). The percentages listed are all percent by volume. The brains were removed and post-fixed in the same fixative (perfusion fix) overnight. The collected brains were subjected to sectioning. Coronal sections were collected (40 μm thick) using the MultiBrain Technology by NeuroScience Associates, Knoxville, Tenn. Sections spanning the cerebellum and hippocampus were maintained in a preservative fixative at −20° C. These sections were subjected to immunohistochemical analysis as described in the subsequent sections.

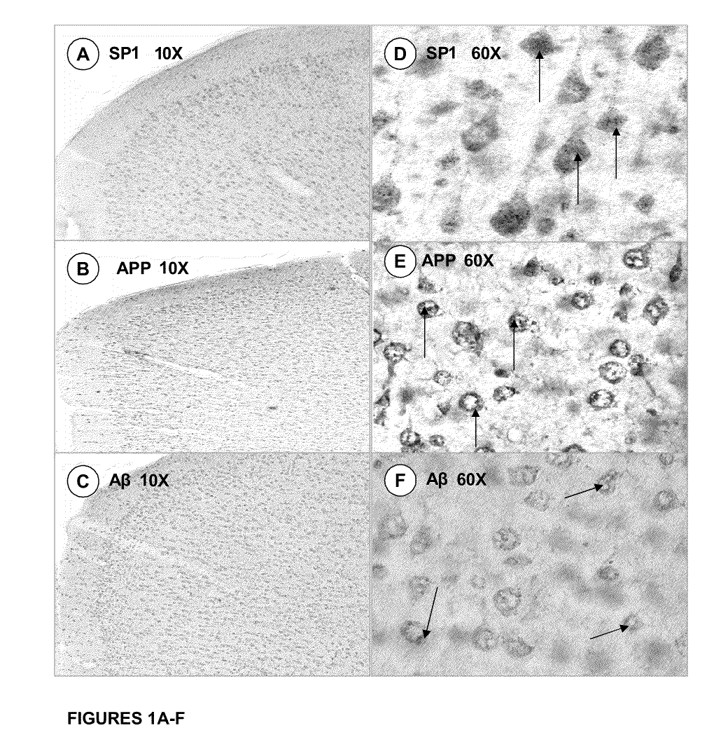

FIGS. 1A-F are micrographs showing SP1, APP and Aβ Immunoreactivity in the Rat Cortex: (A-C). Similarity of staining pattern of SP1, APP and Aβ; (D-F). Cellular characteristics of SP1, APP and AP are at higher magnification. This figure shows that SP1, APP, Aβ are located in the same layers and cells in the cortex region of the brain.

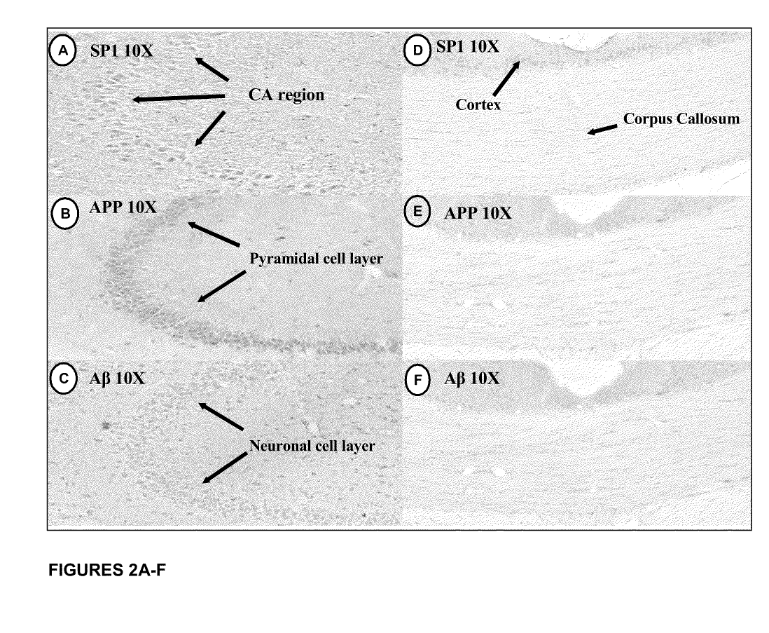

FIGS. 2A-F are micrographs of SP1, APP and Aβ immunoreactivity in hippocampus and corpus callosum of rat. (A-C) show the similarity of staining in the CA region of the Hippocampus; and (D-F) show the absence of staining for SP1, APP and Aβ in Corpus Callosum. These figures show that the three proteins are present in the hippocampus region of the brain which is associated with memory. The figures also show that the three proteins are absent from regions of the brain that do not contain neurons. Therefore the three proteins are residents of neurons which are responsible for information processing in the brain and not in supportive cells.

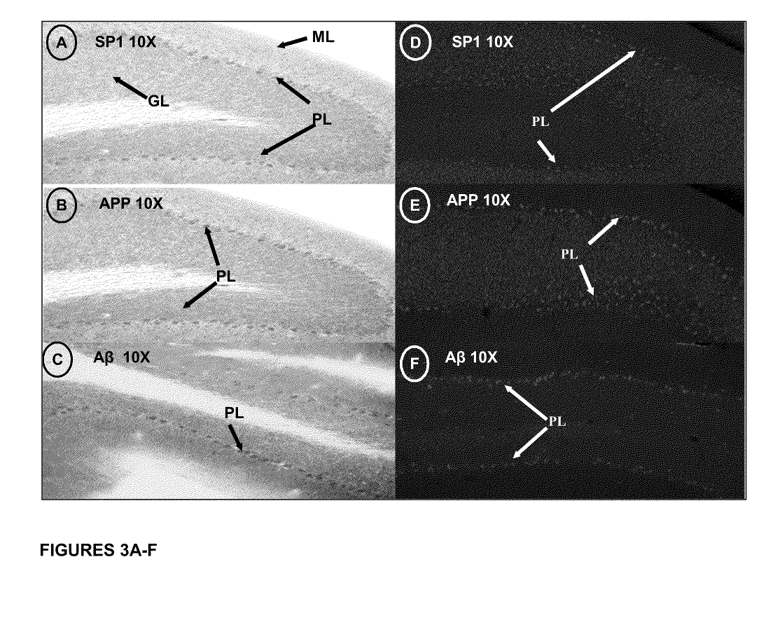

FIGS. 3A-F are micrographs of SP1, APP and Aβ Immunoreactivity in rat cerebellum. (A-C) Show the similarity of SP1, APP, and AB staining in the Purkinje cell layer of the cerebellum; and (D-F) are fluorescent micrographs of the Purkinje cell layer wherein ML=molecular layer; PL=Purkinje cell layer and GL=granular cell layer.

Monkeys: Twenty-three years old Cynomolgus (Macaca fascicularis) female monkeys, housed at the NIH Poolesville, Md. primate facility over the last decade of life, were euthanized with an overdose of pentobarbital, with a veterinary confirmation of death. The brains were excised, cut along the mid-sagittal plane, and each hemisphere was cut into 10 mm sections and rapidly immersion fixed in 10% neutral buffered formalin. The brain sections containing the frontal association cortex were rinsed and taken through a graded series of ethanols, processed, and embedded in paraffin. Eight micron serial sections through the region were collected on charged slides.

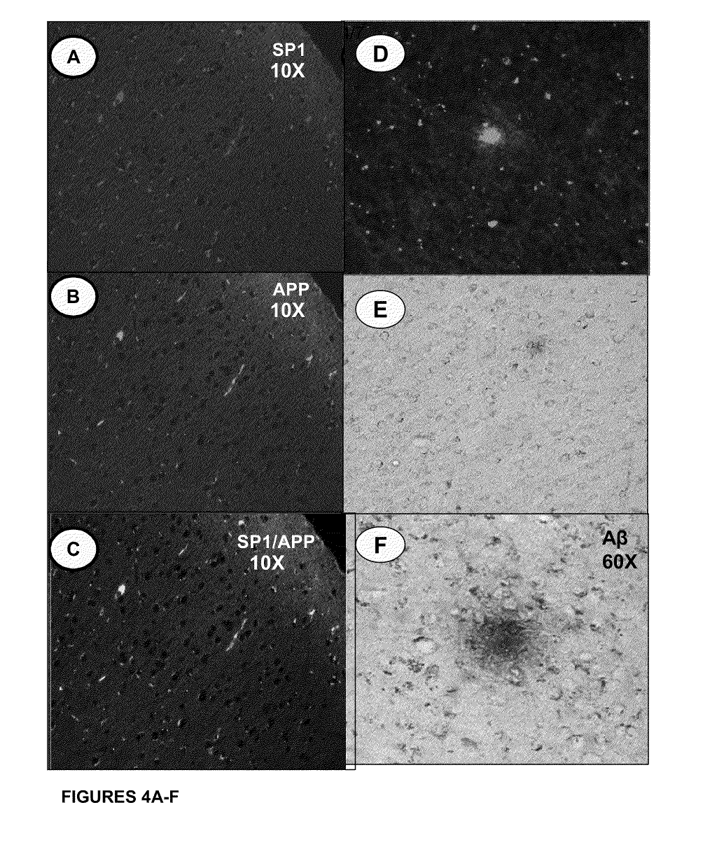

FIGS. 4A-F are photomicrographs of SP1, APP and Aβ Immunoreactivity in Monkey Cortex wherein there is intra-neuronal staining of SP1, APP and Aβ in the frontal association cortex of Cynomolgus monkey (Macaca fascicularis). The fluorescence immuno-reactivity of (A) SP1, (B) APP, and (D) Aβ; (C) shows the co-localization of SP1 and APP; and (E & F) reveals the staining patterns of Aβ in senile plaques. These figures show that the three proteins are present in the same pattern in the cortex of primates. Primates are similar to humans and express plaques. From these figures it is seen that when the figure for SP1 (red) was merged with APP (Green), cells with a yellow coloration were obtained. This suggests that the two proteins are localized to the same neurons. Aβ plaque staining with two different methods can be shown. Neurons that over express SP1 are rich in APP and consequently secrete the amyloidogenic Aβ cleavage product which is associated with plaque formation.

Immunohistochemistry: To determine the cellular distribution of APP, SP1 and Aβ, immunohistochemistry was conducted using free-floating brain tissue of 20 months old rodents and the paraffin embedded brain tissue of 23 years old Cynomolgus monkeys. Free-floating sections of brain tissues obtained from rodents were taken from the preservative fixative and briefly washed in distilled water to rinse away the fixative prior to PBS rinses. Both free-floating rodent and paraffin embedded monkey brain tissues were subjected to brief washes in 1×Phosphate Buffer Saline (PBS) and 3% Hydrogen peroxide. After rinsing, the sections were incubated in PBS with 2% Bovine Serum Albumin (BSA) and 1% Triton X-100 blocking solution for 30 min., and incubated in the presence of primary antibody APP (1:200; Sigma-Aldrich, St. Louis, Mo.), Aβ (1:50; Sigma-Aldrich), SP1 (1:100; Santa Cruz Biotechnology, Santa Cruz, Calif.), overnight at 4° C. Sections were washed with PBS and incubated along with the species-specific biotinylated secondary antibody mouse/rabbit (1:200; Vector Labs, Burlingame, Calif.) for 30 min. The sections were incubated with Streptavidin (Vector Labs) for 30 min, rinsed briefly with PBS, and immunoreactivity detected with the substrate, 3-3′ Diaminobenzidine tetra hydrochloride (DAB) (Vector Labs). The free-floating rodent sections were mounted on microscope slides using procedures provided by Neuroscience Associates, Knoxville, Tenn. Coverslips were mounted with Permanent Mounting Medium (Vector Labs) on the rodent and monkey microslides.

Immunofluorescent staining: Free-floating sections (40 microns) of rat hippocampus, cortex and cerebellum were removed from storage solution, washed with distilled water, 1×Phosphate Buffer Saline (PBS) and 3% Hydrogen peroxide and incubated in 2% Bovine Serum Albumin (BSA)/1% Triton X-100/PBS blocking solution for 30 min. Sections were incubated overnight with the primary antibody for either APP (1:200), SP1 (1:100), or Aβ (1:50), and washed with PBS. Then, the sections were incubated with the species-specific biotinylated secondary antibody against mouse/rabbit (1:200; Vector Labs) for 30 min., and rinsed with buffer. Sections were incubated with Texas Red Streptavidin (1:100; Vector Labs) for 30 min to facilitate the attachment of the fluorescent tag to target proteins. After a brief rinse in PBS, sections were mounted on microscope slides according to standard procedures of Neuroscience Associates. The slides were secured with cover slips using the Fluorescence Mounting Medium (Vector Labs) to preserve the quality of fluorescence immunoreactivity.

Double fluorescence staining was conducted for SP1 and APP in the monkey brain sections. These sections were first immunostained for SP1 and then labeled with Texas Red using the immunofluorescent procedure as described above. After a brief rinse in PBS, the previous steps were repeated using the primary APP antibody labeled with Fluorescein (green) streptavidin (1:200; Vector Labs) to distinguish the presence of APP from SP1. After a brief rinse in PBS, sections were dried and cover-slipped with Fluorescence Mounting Medium (Vector Labs) to preserve the quality of fluorescence immunoreactivity. The double labeling of these proteins SP1 (Texas Red) and APP (Fluorescein) was performed in the same section to show the co-localization in the neurons.

In all cases, negative controls were run on slides from each animal by omitting the primary antibody incubation. No signal was evident after incubation of tissues in secondary antibody alone.

Microscopy: All sections were examined using a Nikon Eclipse E600 microscope with an attached Diagnostic Instruments digital camera using SPOT Diagnostic Instruments system and software (Nikon, Melville, N.Y.). Fluorescent images were viewed using FITC (green) and Rhodamine (red) filters in the Nikon Epi-fluorescence filter set to visualize the immunoreactivity of target proteins. Further studies are needed to quantify the protein expression, cell loss and damage.

Animal Exposure: Adult mice (C57BL/6) were obtained from Jackson labs (Maine). They were divided into 2 groups viz., Con (C), drug treated (D). The drug, toelfenamic acid was dissolved in corn oil and administered at various concentrations from 10 mg/Kg to 100 mg/Kg in corn oil by oral gavage. Control mice were dosed with corn oil only. Treatment was given on alternate days for one month. At the end of the exposure period the animals were decapitated following exposure to CO₂ and hypothermia and various brain regions were isolated and stored at −70° C. This protocol has been approved by the IACUC of the University of Rhode Island.

Western Blot Analysis: APP and Sp1 were obtained from Cell Sciences (Canton, Mass.); Western blotting and quantization procedures were carried out as described in previous published studies (Atwood et al., 1998; Atkins et al., 2003).

siRNA gene silencing: See the published results for the method (Basha et al. 2005)

ELISA Assays: Aβ-40 and Aβ-42 levels were measured using a combination of monoclonal antibody (mAb) 6E10 (specific to an epitope present on 1-16 amino acid residues of Aβ) and R162, and R164 respectively in a double-antibody sandwich ELISA (Mehta et al., 1998; Morishima-Kawashima et al., 2000; Basha et al., 2005).

Statistical treatment: Data was analyzed by two-way analysis of variance (ANOVA) followed by Duncan's or SNK post-hoc test(s) to compare the effects among various treatments and p<0.05 will be considered significant.

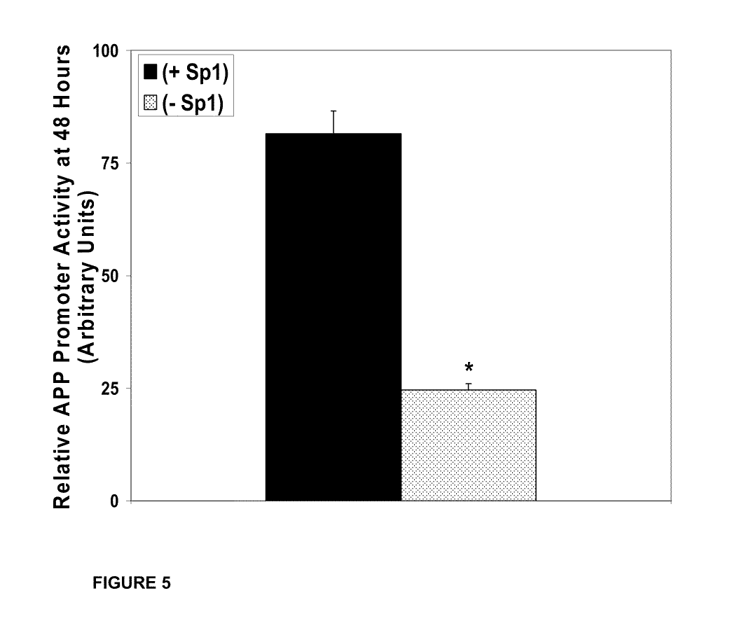

FIG. 5 is a graph of Human APP promoter activity and Gene Silencing (siRNA) in transfected PC12 Cells. This figure shows that when we deplete cells of SP1 using gene silencing techniques (siRNA), and transfect them with the human APP promoter, the activity of this promoter drops dramatically. This is proof that Sp1 drives the APP gene and is a primary activator or driver of the APP gene.

Thus, SP1. APP, and Aβ are co-localized to the same cells in the brain. Neurons that are rich in SP1, are abundant in APP, and are surrounded by secretions of Aβ. The expression of APP in the brain is regulated by Sp proteins. Down-regulation of Sp1 can lower APP and Aβ levels. Additionally, Tolfenamic acid lowers SP1, APP, and Aβ levels. Thus, Drugs like Tolfenamic acid that induce degradation of Sp1 represent a new class of disease-modifying drugs for the treatment of AD.

The foregoing description has been limited to a specific embodiment of the invention. It will be apparent; however, that variations and modifications can be made to the invention, with the attainment of some or all of the advantages of the invention. Therefore, it is the object of the appended claims to cover all such variations and modifications as come within the true spirit and scope of the invention.

Having described our invention, what I now claim is:

1. A method of treating Alzheimer's disease in a patient, said method comprising administrating tolfenamic acid to said patient.

2. The method of claim 1, wherein the said treatment modulates precursors of pathogenic intermediates.

3. The method of claim 2, wherein the treatment can prevent patients from developing Alzheimer's disease.

4. The method of claim 1, wherein the treatment lowers pathogenic endpoints by 25-50%.

5. The method of claim 1, wherein the treatment lowers cerebral Sp1, APP and Aβ levels.
